(19) United States
(12) Patent Application Publication
(10) Pub. No.: US 2020/0308279 A1
(43) Pub. Date: Oct. 1, 2020

(54) CHIMERIC ANTIGEN RECEPTORS

(71) Applicant: OSLO UNIVERSITETSSYKEHUS HF, Oslo (NO)

(72) Inventors: Sébastien WÄLCHLI, Oslo (NO); Even WALSENG, Oslo (NO); Else Marit INDERBERG, Oslo (NO); Hakan KÖKSAL, Oslo (NO)

(21) Appl. No.: 16/753,948

(22) PCT Filed: Mar. 5, 2018
(86) PCT No.: PCT/IB2018/000303

§ 371 Date: Apr. 6, 2020

Related U.S. Application Data
(60) Provisional application No. 62/568,837, filed on Oct. 6, 2017.
(60) Provisional application No. 62/583,058, filed on Nov. 8, 2017.

(51) Int. Cl. C07K 16/28; C07K 14/705; C12N 5/0783; A61K 35/17

(52) U.S. Cl. CPC C07K 16/2809; C07K 14/70521; C07K 16/2833; C07K 2319/01; A61K 35/17; C07K 2319/03; C07K 2317/565; C12N 5/0636

(57) ABSTRACT

Provided herein are compositions and methods for immunotherapy. In particular, provided herein are chimeric antigen receptors, cells expressing chimeric antigen receptors, and use of such cells in immunotherapy (e.g., cancer immunotherapy).

### CROSS-REFERENCE TO RELATED APPLICATIONS

This application claims priority to and the benefit of U.S. Provisional Application No. 62/568,837, filed Oct. 6, 2017 and U.S. Provisional Application No. 62/583,058, filed Nov. 8, 2017, which are hereby incorporated by reference in their entireties.

### FIELD OF THE INVENTION

Provided herein are compositions and methods for immunotherapy. In particular, provided herein are chimeric antigen receptors, cells expressing chimeric antigen receptors, and use of such cells in immunotherapy (e.g., cancer immunotherapy).

### BACKGROUND OF THE INVENTION

Immunotherapy connecting the power of T cells and redirecting them against tumours has in the past 5 years proven very successful and attracted considerable interest. It includes the redirection of effector cells (mainly T cells and natural killer cells) with selected antigen receptors. To date, two main redirecting agents have been developed: Chimeric Antigen Receptors (CARs) based on antigen binding domains from antibodies; and T cell Receptors (TCRs). Antibodies, being soluble proteins, are modified into cellular receptors by (i) fusing antigen binding domains to resident protein transmembrane (TM) domains and (ii) adding signalling domain of known TCR signalling proteins, mainly phosphorylation sites of partners involved in signal I and II (Letourneur, F. & Klausner, T-cell and basophil activation through the cytoplasmic tail of T-cell-receptor zeta family proteins. Proc. Nat'l Acad. Sci. 88, 8905-8909 (1991); Romeo, C. & Seed, B. Cellular immunity to HIV activated by CD4 fused to T cell or Fc receptor polypeptides. Cell 64, 1037-1046 (1991); Irving, B. A. & Weiss, A. The cytoplasmic domain of the T cell receptor zeta chain is sufficient to couple to receptor-associated signal transduction pathways. Cell 64, 891-901 (1991)). The composition and combination of domains linked to the single chain variable part of the antibody (scFv) are diverse and no clear road map of the most potent universal design has been drawn so far. These CARs have the capacity to generate an immune synapse and trigger effector cell functions, cytokine release and target killing. After the astonishing results generated by different teams using anti-CD19 CAR for the treatment of haematological malignancies (Jensen, M. C. & Riddell, S. R. Design and implementation of adoptive therapy with chimeric antigen receptor-modified T cells. Immunol Rev 257, 127-144, doi:10.1111/imr.12139 (2014); Brentjens, R. J. et al. CD19-targeted T cells rapidly induce molecular remissions in adults with chemotherapy-refractory acute lymphoblastic leukemia. Sci Transl Med 5, (2013); Kochenderfer, J. N. & Rosenberg, S. A. Treating B-cell cancer with T cells expressing anti-CD19 chimeric antigen receptors. Nat Rev Clin Oncol 10, 267-276, (2013); Kalos, M. et al. T cells with chimeric antigen receptors have potent antitumor effects and can establish memory in patients with advanced leukemia. Sci Transl Med 3, 95ra73, (2011)), the use of these constructs has had a meteoric rise. New targets are presently evaluated, but the outcome, in particular when dealing with solid tumours, was not as successful as observed with the common B-cell marker CD19 (Park, J. R. et al. Adoptive transfer of chimeric antigen receptor re-directed cytolytic T lymphocyte clones in patients with neuroblastoma. Mol Ther 15, 825-833, (2007); Lamers, C. H. et al. Treatment of metastatic renal cell carcinoma with CAIX CAR-engineered T cells: clinical evaluation and management of on-target toxicity. Mol Ther 21, 904-912, (2013); Katz, S. C. et al. Phase I Hepatic Immunotherapy for Metastases Study of Intra-Arterial Chimeric Antigen Receptor-Modified T-cell Therapy for CEA+ Liver Metastases. Clin Cancer Res 21, 3149-3159, (2015)).

Solid tumors present a different set of challenges compared to B cell malignancies: overall lesser sensitivity to T cell mediated cytotoxicity, a microenvironment that presents with an array of immunosuppressive mechanisms differing between tumor types, and a paucity of target antigens with an expression profile as favorable as CD19. Despite an impressive number of investigated targets, few target candidates are tumor-specific, or restricted to the tumor and a “dispensable” normal cell type or a tissue that is sheltered from an immune attack. In this perspective, identifying valid targets to achieve efficacious tumor rejection while ensuring patient safety is an essential goal. Therefore, an obvious bottleneck in CAR therapy is the lack of cancer-specific targets. Indeed, when introduced into T cells, conventional CARs are limited to antigens (proteins, sugar residues) expressed on the surface of the target cells.

The second type of receptors, TCRs, is not limited to the detection of surface antigens like antibodies and conventional CARs. Rather they are defined as “obsessed” with peptides presented on the MHC molecules, pMHC (Yin, L., et al., T cells and their eons-old obsession with MHC. Immunol Rev 250, 49-60, (2012)). The human MHC is also called the HLA (human leukocyte antigen) complex (often just the HLA). Considering that all the proteins expressed by a given cell may be degraded and loaded onto an MHC molecule, TCRs can potentially recognize the whole proteome. This represents a striking numerical advantage over conventional CARs in terms of possible targets. In addition, TCRs can be specifically directed against a mutant variant of a protein and spare the wild type form (Parkhurst, M. R. et al. Isolation of T cell receptors specifically reactive with mutated tumor associated antigens from tumor infiltrating lymphocytes based on CD137 expression. Clin Cancer Res. (2016)); hence the TCR can distinguish cancer cells expressing the mutated protein from healthy cells expressing the non-mutated protein. On the other hand, TCRs are complicated molecules to manipulate: they are heterodimers composed of an α- and a β-chain, they do not signal by themselves but require a battery of signalling proteins associated to recruit all the components to create an immune synapse. In addition, their localization at the plasma membrane depends on the CD3 complex, whose expression is restricted to T cells. Consequently TCR-based redirection has only been available in T cells since they are the only cells that possess all components required for proper TCR stimulation. In addition, the exogenous TCR might compete with the endogenous TCR for the use of these signalling proteins (Ahmadi, M. et al. CD3 limits the efficacy of TCR gene therapy in vivo. Blood 118, 3528-3537, (2011)). Another issue with the introduction of a second TCR into the redirected T cell is the possibility to form mixed dimers thus generating novel TCRs (van Loenen, M. M. et al. Mixed T cell receptor dimers harbor potentially harmful neoreactivity. Proc. Nat'l Acad. Sci. 107, 10972-10977, (2010); Bendle, G. M. et al. Lethal graft-versus-host disease in mouse models of T cell receptor gene therapy. Nat Med 16, 565-570, 561p following 570, (2010)). Although mispairing of TCRs has yet to be observed in a clinical setting, an important number of innovations has been developed in order to prevent this. The addition of extra cysteines on the constant part of both chains represented the first step to support the pairing of the redirecting TCR (Cohen, C. J. et al. Enhanced antitumor activity of T cells engineered to express T-cell receptors with a second disulfide bond. Cancer Res 67, 3898-3903 (2007); Kuball, J. et al. Facilitating matched pairing and expression of TCR chains introduced into human T cells. Blood 109, 2331-2338, (2007). Another strategy was to replace the constant domains of the therapeutic TCRs with murine constant domains (Sommermeyer, D. & Uckert, W. Minimal amino acid exchange in human TCR constant regions fosters improved function of TCR gene-modified T cells. J. Immunol. 184, 6223-6231, (2010); Bialer, G., et al., Selected murine residues endow human TCR with enhanced tumor recognition. J. Immunol. 184, 6232-6241, (2010)). The rationale behind this was (i) mouse TCR constant domain has higher affinity to human CD3 than human constant domain (Cohen, C. J., et al., Enhanced antitumor activity of murine-human hybrid T-cell receptor (TCR) in human lymphocytes is associated with improved pairing and TCR/CD3 stability. Cancer Res. 66, 8878-8886, (2006)) and (ii) this would increase the chance of the correct TCRs pairing, accepting per se that xenogenous pairing would not occur. However, to our knowledge mouse and human constant parts have never been shown not to pair. Although these modifications might improve TCR expression and signalling of certain TCRs, but not universally (Kuball et al., supra; Sommermeyer et al., supra; Bialer et al., supra), one cannot exclude that the higher affinity of mouse TCR constant domain for the human CD3 could be the main mechanism behind this improved effect (Cohen et al., supra). Thus the CD3 monopolization seems to represent the major factor improving TCR redirection observed with murinized constructs. The use of murine protein domain in a therapeutic construct might lead to rejection by the patient's immune system as previously reported with a non-humanized CAR (Maus, M. V. et al. T cells expressing chimeric antigen receptors can cause anaphylaxis in humans. Cancer Immunol. Res. 1, 26-31 (2013)). Finally, another strategy to improve redirected TCR potency and avoiding the mispairing was by fusing of signalling components to the intracellular domain of one of the TCR chains (Govers, C. et al. TCRs genetically linked to CD28 and CD3epsilon do not mispair with endogenous TCR chains and mediate enhanced T cell persistence and anti-melanoma activity. J. Immunol. 193, 5315-5326, (2014)).

According to Aggen et al, Gene Ther. 2012 April; 19(4): 365-374, several labs have attempted to use single-chain, three-domain TCRs (VαVβCβ) that can mediate proximal signalling through fused intracellular signalling domains. However, success has been limited by low surface expression level of the TCRs and the constructs do not avoid the risk of mispairing with endogenous TCR chains. Aggen et al teach that three domain TCR constructs (VαVβCβ) yield mispaired receptors in the presence of an endogenous α chain because of the contained Cβ domain. However, stabilized two domain TCRs (VαVβ) with chimeric signalling domains avoid mispairing altogether and mediate T cell activity.

Thus, improved constructs and methods for immunotherapy are needed.

### SUMMARY OF THE INVENTION

Provided herein are compositions and methods for immunotherapy. In particular, provided herein are chimeric antigen receptors, cells expressing chimeric antigen receptors, and use of such cells in immunotherapy (e.g., cancer immunotherapy).

The present disclosure provides novel chimeric antigen receptors (CARs) with specific affinity for HLA complexes presenting peptides. Such TCR-CARs may recognize a wider range of targets than conventional CARs relying on antigen binding domains obtained from antibodies. In some embodiments, the CARs are expressed by immune effector cells and convey targeted cytotoxicity. In some embodiments, an antigen binding domain comprising two sequences, each comprising a variable domain and a constant domain, is a robust alternative to various scFvs. Without being bound by theory, the stability may be increased when the two sequences in the antigen binding domain are connected by more than one disulfide bridge formed by cysteine residues in the constant domains. The substitutions disclosed herein have minimal risk of introduction of undesired epitopes. It is known that conventional TCRs may bind their target with lower affinity, but higher specificity than antibodies. Accordingly, the CARs herein provide a broader target range with lower risk of undesired immunologic responses compared to CARs relying on non-human or humanized antibody fragments. Furthermore, when the two sequences in the antigen binding domain are separated by a 2A ribosomal skipping sequence, equimolar production can be achieved and contribute to their desired dimerization. Mispairing is avoided by the use of a single transmembrane domain in the CAR. The CARs described herein are functional and may act independently of the endogenous TCR signalling machinery (e.g., the CD3 complex). This represents a great advantage over classical overexpression of full-length TCRs as it also means that the CARs are functional in other cells than T cells. Notably, in some embodiments, the CARs herein are especially valuable in combination with conventional CARs targeting surface epitopes via antigen binding domains from antibodies. For example, it is contemplated that, in some embodiments, if the tumour cells develop resistance to the conventional CAR therapy by hiding the surface antigen, the TCR-CAR is still be able to target the tumour cells. In some embodiments, such combinations utilize immune effector cells expressing both the TCR-CAR and a conventional antibody based CAR. In some embodiments, a combination therapy comprises a pharmaceutical composition comprising immune effector cells expressing the TCR-CAR and immune effector cells expressing conventional antibody based CAR and administration of such pharmaceutical compositions comprising immune effector cells expressing the TCR-CAR before, simultaneously or subsequently of administration of a pharmaceutical composition comprising immune effector cells expressing a conventional antibody based CAR.

In one embodiment, a chimeric antigen receptor is provided comprising a) an extracellular antigen binding domain with specific affinity for HLA complexes presenting peptides (e.g., obtained from a tumor reactive TCR); b) a transmembrane domain c) an intracellular signalling domain; wherein the antigen binding domain comprises two sequences (polypeptides), each comprising a variable domain and a constant domain; wherein the antigen binding domain is connected to a single transmembrane domain. In some embodiments, the two polypeptides in the antigen binding domain are connected by at least one disulfide bridge formed by cysteine residues in the constant domains. In some embodiments, the two polypeptides in the antigen binding domain are connected by more than one disulfide bridge formed by cysteine residues in the constant domains. In some embodiments, the two polypeptides in the antigen binding domain are connected by two disulfide bridges each formed by two cysteine residues in the constant domains. In some embodiments, the transmembrane domain comprises or consists of SEQ ID NO:5 or sequences at least 90% identical to said sequence (e.g., provided any difference is in the form of conservative substitutions). In some embodiments, the intracellular signalling domain comprises or consists of SEQ ID NO:6 or sequences at least 90% identical to said sequence (e.g., provided any difference is in the form of conservative substitutions). In some embodiments, the intracellular signalling domain comprises or consists of SEQ ID NO:6 and SEQ ID NO:7. In some embodiments, the antigen binding domain comprises or consists of SEQ ID NO:1 and SEQ ID NO:2 or 16, or functional fragments thereof. In some embodiments, the antigen binding domain comprises or consists of SEQ ID NO:1 and SEQ ID NO:2 or 16, or sequences with at least 95% identity thereof. In some embodiments, the antigen binding domain comprises or consists of SEQ ID NO:1 and SEQ ID NO:2 or 16, or sequences with at least 95% identity thereof, provided SEQ ID NO:1 comprises the three CDRs DSVNN, IPSGT and AVNAGNMLTF and provided SEQ ID NO:2 or 16 comprises the three CDRs MDHEN, SYDVKM and ASSSGVTGELFF. In some embodiments, the antigen binding domain comprises or consists of SEQ ID NO:8 and SEQ ID NO:9 or 17, or functional fragments thereof. In some embodiments, the antigen binding domain comprises or consists of SEQ ID NO:8 and SEQ ID NO: 9 or 17, or sequences with at least 95% identity thereof. In some embodiments, the antigen binding domain comprises or consists of SEQ ID NO:8 and SEQ ID NO:9 or 17, or sequences with at least 95% identity thereof, provided SEQ ID NO:8 comprises the three CDRs DRGSQS, IYSNGD and AVNFGGGKLIF and provided SEQ ID NO:9 or 17 comprises the three CDRs MRHNA, SNTAGT and ASSLSFGTEAFF.

In certain embodiments, a chimeric antigen receptor is provided comprising a) an extracellular antigen binding domain with specific affinity for HLA complexes presenting peptides; b) a transmembrane domain; c) an intracellular signalling domain; wherein the antigen binding domain comprises two sequences, each comprising a variable domain and a constant domain; wherein the antigen binding domain is connected to a single transmembrane domain; and wherein the antigen binding domain comprises one constant domain from an alpha chain and one constant domain from a beta chain.

In one embodiment, a chimeric antigen receptor is provided comprising a) an extracellular antigen binding domain with specific affinity for HLA complexes presenting peptides; b) a transmembrane domain c) an intracellular signalling domain; wherein the antigen binding domain comprises two polypeptides, each comprising a variable domain and a constant domain, wherein the two polypeptides in the antigen binding domain are connected by two disulfide bridges each formed by two cysteine residues in the constant domains; wherein the antigen binding domain is connected to a single transmembrane domain.

In some embodiments, a chimeric antigen receptor is provided comprising a) an extracellular antigen binding domain with specific affinity for HLA complexes presenting peptides; b) a transmembrane domain; c) an intracellular signalling domain; wherein the antigen binding domain comprises two sequences, each comprising a variable domain and a constant domain; wherein the antigen binding domain is connected to a single transmembrane domain; and wherein the antigen binding domain comprises one constant domain represented by SEQ ID NO:3 and one constant domain represented by SEQ ID NO:4.

In one embodiment, a chimeric antigen receptor is provided comprising: a) an extracellular antigen binding domain with specific affinity for HLA complexes presenting peptides; b) a transmembrane domain; c) an intracellular signalling domain; wherein, the antigen binding domain comprises two sequences, each comprising a variable domain and a constant domain; wherein, the antigen binding domain is connected to a single transmembrane domain; and wherein the antigen binding domain comprises one constant domain represented by SEQ ID NO:3 or sequences with more than 98% identity thereto provided the amino acid residues in position 48 and 91 are both cysteine residues and one constant domain represented by SEQ ID NO:4 or sequences with more than 98% identity thereto provided the amino acid residues in position 57 and 131 are both cysteine residues. In one exemplary embodiment, a chimeric antigen receptor is provided comprising:

a) an extracellular antigen binding domain with specific affinity for HLA complexes presenting peptides; b) transmembrane domain; c) an intracellular signalling domain; wherein the antigen binding domain comprises two sequences, each comprising a variable domain and a constant domain; wherein, the antigen binding domain is connected to a single transmembrane domain; and wherein the antigen binding domain comprises one constant domain represented by SEQ ID NO:3 or sequences with more than 98% identity thereto provided the amino acid residue in position 48 is a cysteine residue and one constant domain represented by SEQ ID NO:4 or sequences with more than 98% identity thereto provided the amino acid residue in position 57 is a cysteine residue.

In further embodiments, a chimeric antigen receptor is provided comprising a) an extracellular antigen binding domain with specific affinity for HLA complexes presenting peptides; b) a transmembrane domain; c) an intracellular signalling domain; wherein the antigen binding domain is connected to a single transmembrane domain; wherein the antigen binding domain comprises a constant domain from an alpha chain and a constant domain from a beta chain; wherein the threonine residue in position 48 in the constant domain from the alpha chain is substituted with a cysteine residue and wherein the serine residue in position 57 in the constant domain from the beta chain is substituted with a cysteine residue.

In further embodiments, a nucleic acid is provided encoding a receptor described herein. In some embodiments, the two sequences comprising a variable domain and a constant domain are separated by a 2A ribosomal skipping sequence. In particular, SEQ ID NO:1 and SEQ ID NO:2 or 16 may be separated by a 2A ribosomal skipping sequence or SEQ ID NO:8 and SEQ ID NO:9 or 17 may be separated by a 2A ribosomal skipping sequence.

In yet other embodiments, a cell is provided expressing the receptors according to the first embodiment in its cell membrane. In some embodiments, the cell is a T cell, a natural killer cell, an immortalized cell line or another cell type (e.g., a cell not associated with the immune system). In some embodiments, the cell is a NK-92 cell.

Also provided is a method for stimulating a lymphocyte (e.g., T cell or natural killer cell) mediated immune response to a target cell population or tissue in a subject, the method comprising administering to a subject an effective amount of a cell described herein. In some embodiments, the target cell population or tissue is a cancer cell or tumor.

Additionally provided is a method of treating cancer in a subject, comprising: administering to the mammal an effective amount of a cell described herein. In some embodiments, the cell is an autologous T cell.

Also provided herein is the use of a cell described herein to stimulate a T cell-mediated immune response to a target cell population or tissue in a subject.

Still other embodiments provide the use of a cell described herein to treat cancer in a subject.

Other embodiments provide a cell or pharmaceutical composition comprising a cell described herein for use in treating cancer or stimulating a T cell-mediated immune response to a target cell population or tissue in a subject.

Additional embodiments are described herein.

### BRIEF DESCRIPTION OF THE DRAWINGS

FIG. 1. Design of the TCR-CAR constructs. (a) TCR-CAR gene design. TCRα and β chain were truncated at the level of their transmembrane region (TM) or domain, cysteines were added on their constant domains and the two chains were linked by a 2A peptide sequence. (b) sTCR was produce as a soluble protein which, probably following the vesicular secretion pathway, was released in the cellular medium (left). Correct folding should ensure specific binding to a peptide-MHC (pMHC) complex and signal transduction through CD28-CD3 signalling tail (right).

FIG. 2. Membrane expression of TCR-CAR. (a) DMF5 TCR and TCR-CAR were expressed in J76 cell line. (b) Same as in (a) but Radium-1 TCR (plain) and TCR-CAR (dotted) were expressed and J76 cells were stained with anti-Vb3 antibody (Vb). (c) The cells as in A and B were stained with anti-CD3 antibody.

FIG. 3. Functional activity of TCR-CAR. (a) Primary peripheral T cells isolated from a healthy donor were mock electroporated or electroporated with mRNA encoding Radium-1 or DMF5 constructs. (b) The same cells were co-incubated 18 hours later with APCs loaded with the indicated peptides for 5 hours (grey=no peptide, white=TGFbR2 peptide and black=MART-1 peptide). (c) same as in (b) but here Radium-1 TCR-CAR was compared to the full-length Radium-1 TCR. DMF5 was included as a control for M1 loading. (d) DMF5 (black) or Radium-1 (grey) TCR-CAR and full-length constructs expressing T cells were analyzed for the indicated cytokine response in the CD8 population. (e) same as in (d) where specific lysis of target cells loaded with the indicated peptide was analyzed.

FIG. 4. TCR-CAR can redirect NK-92 cells. (a) NK-92 cells were non transfected (grey) or transfected with DMF5 TCR-CAR. (b) Stimulation of plain NK-92 cells (white) or transfected with TCR-CAR constructs (DMF5, black and Radium-1, grey) with Granta-519 loaded (+) or not (−) with the cognate peptide was performed for 6 hours at a E:T ratio of 1:2. (c) Specific lysis of target cells loaded with the indicated peptide (MART-1 peptide, black, TGFbR2 peptide, white, no peptide, grey) by plain NK-92 (circles) or NK-92 expressing TCR-CAR (squares) at different E:T ratios in a BLI cytotoxic assay.

FIG. 5. Expression analysis of Radium-1 TCR and TCR-CAR J76 and NK-92 cells were electroporated with water (grey), Radium-1 TCR mRNA (solid line) or Radium-1 TCR-CAR mRNA (dashed line).

FIG. 6. (top) shows the structure, not to scale, of a naturally occurring αβ TCR including its most important domains. FIG. 6 (bottom) visualizes the structure, not to scale, of a TCR-CAR according to the two experiments herein.

FIG. 7. shows how the constant domain of an alpha chain represented by SEQ ID NO:3 may be connected to the constant domain of a beta chain represented by SEQ ID NO:4

FIG. 8 shows sequences of exemplary CAR constructs.

### DEFINITIONS

As used herein, the term “tumor reactive TCR” refers to any TCR that has specific affinity for tumor or cancer cells and low or no affinity for non-cancerous cells. In some embodiments, antigen binding domains from tumor reactive TCRs have “specific affinity for HLA complexes presenting peptides.”

As used herein, “specific affinity for HLA complexes presenting peptides” refers to measurable and reproducible interactions with the target in the presence of a heterogeneous population of molecules and/or cells. An antigen binding domain specific affinity for HLA complexes presenting peptides binds its target with greater affinity, avidity, more readily, and/or with greater duration than it binds to other targets. In particular, an antigen binding domain with specific affinity for HLA complexes presenting peptides will have low binding of other targets under physiological conditions. In particular, T cells expressing CARs with specific affinity for HLA complexes presenting peptides may provide significant killing of cells expressing the peptide-loaded HLA complex (e.g., a HLA complex presenting a peptide) under physiological conditions, but low killing of other cells (e.g., cells expressing HLA complexes without the target peptide). As used herein, “physiological conditions” means any in vitro or in vivo condition suitable for growth, proliferation, propagation and/or function of human cells, for example neutral aqueous buffer solutions at 37° C.

As used herein, “transmembrane domain”, refers to the part of a CAR that is generally embedded in the cell membrane when expressed by an immune effector cell. The present invention is not limited to particular transmembrane domains. Several suitable transmembrane domains may be utilized. The transmembrane domain may span the membrane one or multiple times. Accordingly, when an antigen binding domain is connected to a single transmembrane domain, it means that only one of the two sequences, i.e. only one of the two peptide chains, in the antigen binding domain is connected to a transmembrane domain. In some exemplary embodiments, the transmembrane domain is connected to the C-terminal of a constant domain derived from a beta chain.

As used herein “intracellular signalling domain,” refers to the part of a CAR located inside the immune effector cell when the CAR is expressed in the cell membrane of an immune effector cell. This domain participates in conveying the signal upon binding of the target. The signal may contribute to activation, cytokine production, proliferation and/or cytotoxic activity. The present invention is not limited to particular intracellular signaling domains. Examples include, but are not limited to, signalling domains from CD28, CD3, 4-1BB, OX40, ICOS etc. or functional variants/fragments of such domains.

As used herein, “extracellular domain”, refers to the part of a CAR facing the extracellular environment when expressed in the cell membrane of cell (e.g., an immune effector cell). In some embodiments, the extracellular domain comprises an antigen binding domain. In some embodiments, the antigen binding domain comprises antigen binding domains obtainable from naturally occurring TCRs or synthetic TCRs (e.g., tumor reactive TCRs). In particular, the antigen binding domain may comprise a “soluble TCR”-construct (sTCR). In particular, the antigen binding domain may consist of a “soluble TCR” (sTCR) e.g., a heterodimer comprising one variable domain and one constant domain from an alpha chain, and one variable domain and one constant domain from a beta chain.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab).sub.2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. .kappa. and .lamda. light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

As used herein, the term “autologous” is meant to refer to any material derived from the same individual to which it is later to be re-introduced into the individual.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

“Co-stimulatory ligand,” as the term is used herein, includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class 1 molecule, BTLA and a Toll ligand receptor.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-.beta., and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class 1 molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). Examples of subjects include humans, dogs, cats, mice, rats, and transgenic species thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### DETAILED DESCRIPTION OF THE INVENTION

Provided herein are compositions and methods for immunotherapy. In particular, provided herein are chimeric antigen receptors, cells expressing chimeric antigen receptors, and use of such cells in immunotherapy (e.g., cancer immunotherapy).

The present disclosure concerns chimeric antigen receptors and cells expressing them. Provided herein are membrane bound TCRs built on a technique validated to produce soluble TCRs (sTCR) (Tadesse, F. G. et al. Unpredicted phenotypes of two mutants of the TcR DMF5. J Immunol. Methods 425, 37-44, (2015); Walseng, E. et al. Soluble T-cell receptors produced in human cells for targeted delivery. PLoS One 10, e0119559, (2015)).

In contrast to most antibodies, TCRs often bind their pMHC target with fairly low affinity (KD˜0.1-500 μm). Thus, CAR technologies based on scFvs cannot automatically be transferred to antigen binding domains from TCRs. In addition, the constructs described herein utilize variable and constant regions from alpha and beta chains of a TCR linked to a single transmembrane and signalling domain.

Furthermore, upon binding of its pMHC target, TCRs may trigger downstream events in a unique way. As described by Brazin et al, Front Immunol. 2015; 6: 441.: “Direct evidence that the TCR acts as a mechanosensor was experimentally shown [ . . . ] where mere binding without force was insufficient for triggering, but tangential force led to T cell activation. The concept of the T cell acting as a mechanosensor may reconcile the discrepancy between the precision in recognition described above and low affinity of free unbound ligand”. Thus, providing a chimeric receptor construct comprising an antigen binding domain from a TCR, able to retain its specificity and convey a signal into both T cells and NK-cells, and described herein, was not trivial and was unexpected.

Accordingly, in some embodiments, the present invention provides a construct comprising a sTCR construct (Walseng et al., supra) linked to the transmembrane and signalling domains of a CAR construct, namely CD28 TM followed by part of CD28 and CD3 intracellular domains (Almasbak, H. et al. Inclusion of an IgG1-Fc spacer abrogates efficacy of CD19 CART cells in a xenograft mouse model. Gene Ther. 22, 391-403, (2015)). Experiments described herein used two therapeutic TCRs: DMF5, a MELAN-A peptide specific TCR (Johnson, L. A. et al. Gene transfer of tumor-reactive TCR confers both high avidity and tumor reactivity to nonreactive peripheral blood mononuclear cells and tumor-infiltrating lymphocytes. J. Immunol. 177, 6548-6559 (2006)) and Radium-1 TCR, a TCR targeting a TGF beta Receptor 2 (TGFbR2) frameshift mutation (Inderberg, E. M. et al. T cell therapy targeting a public neoantigen in microsatellite instable colon cancer reduces in vivo tumor growth. Oncoimmunology 6, e1302631, (2017)). Both TCR-CARs were constructed and expressed. Radium-1 TCR-CAR was well detected and could also be seen in a CD3-free system such as the NK cell line, NK-92. Both TCR-CARs could redirect T cells and NK cells against their cognate pMHC, and trigger target cell killing. Thus TCR-CAR provides an alternative to redirect effector cells and render non-T cells pMHC-restricted, opening the CAR targeting to the whole proteome.

Exemplary constructs and uses are described below.

### I. CAR-TCRs

Provided herein are constructs comprising a soluble TCR construct fused to a CAR-signalling tail via a transmembrane domain. A TCR is a heterodimeric transmembrane protein (e.g. TCR α/β) that recognizes a peptide in the context of a MHC/HLA molecule. The present disclosure describes the expression of a modified version of a natural TCR where the transmembrane (TM) domain is changed by substituting the cytoplasmic and TM domains of one of the TCR chains (e.g., TCR β) with a chimeric signalling domain.

The present invention is not limited to particular TCR constructs, transmembrane domains, or intracellular signaling domains. Exemplary components are described herein.

The present invention is not limited to particular antigen binding domains from TCRs. In some embodiments, antigen binding domains are based on tumor reactive TCRs. Examples include, but are not limited to, TCRs specific for Radium 1 (WO2017194555; herein incorporated by reference in its entirety), HER2, DMF5, and those described in WO2017203370A2, WO2017197347A1 (delta-1), WO2017195153A1 (CT45), WO2017194555A1 (TGF3RII), WO2017194924A1, US20170319638A1, WO2017189254A1 (WO2017194555), WO2017174823A1 (B2), WO2017174824A1(B2), WO2017174822A1(B2), U.S. Pat. No. 9,822,162B2 (HPV16E6), U.S. Pat. No. 9,717,758B2 (DMF5), U.S. Pat. No. 9,487,573B2 (NY-ESO-1), EP2831109B1 (gamma9-T-cell and delta2-T-cell), U.S. Pat. No. 9,345,748B2, U.S. Pat. No. 9,678,061B2, U.S. Pat. No. 8,283,446B2 (human leukocyte antigen serotype A1), U.S. Pat. No. 9,181,527B2, U.S. Pat. No. 8,519,100B2 (HLA-A2), U.S. Pat. No. 9,688,739B2, U.S. Pat. No. 8,697,854B2 (tyrosinase), U.S. Pat. No. 8,431,690B2, U.S. Pat. No. 9,315,865B2 (Wilm's tumor), U.S. Pat. No. 8,613,932B2 (gp100), U.S. Pat. No. 9,128,080B2, U.S. Pat. No. 9,133,264B2, U.S. Pat. No. 8,217,009B2, EP2016102B1, U.S. Pat. No. 8,951,510B2 (MAGE-A4), U.S. Pat. No. 8,217,144B2, U.S. Pat. No. 8,017,730B2 (HLA-A24), U.S. Pat. No. 7,915,036B2, U.S. Pat. No. 8,361,794B2, EP1758935B1, U.S. Pat. No. 9,512,197B2 (NY-ESO-1), U.S. Pat. No. 7,951,783B2 (Wilm's tumor), U.S. Pat. No. 7,541,035B2, U.S. Pat. No. 7,666,604B2, U.S. Pat. No. 7,538,196B2 (CD28), U.S. Pat. No. 7,456,263B2 (p53), U.S. Pat. No. 7,723,111B2, and U.S. Pat. No. 6,770,749B2; each of which is herein incorporated by reference in its entirety.

The majority of naturally occurring TCRs are heterodimers comprising an alpha (α) chain and a beta (β) chain. Most alpha and beta chains comprise an N-terminal variable domain with three Complementarity-determining regions (CDRs). The CDRs are generally sensitive for modifications, however, some modification of the framework sequences, e.g., the sequences flanking the CDRs, is believed to be tolerated without adversely affecting the function of an antigen binding domain. In general, alpha chains and beta chains are encoded and synthesized in an immature form endogenously, with a leader sequence located N-terminal (i.e. upstream) of the variable domain. The leader sequences, also known as signal peptides, facilitate membrane localization of the polypeptide chains. The leader sequences are usually cleaved from the alpha chains and beta chains upon insertion into the cell membrane. Accordingly, the leader sequences are not generally present in the TCR-CARs when they are expressed in the cell membrane. In some embodiments, the expression constructs described herein, i.e. the nucleic acids, encode leader sequences. In particular, in some embodiments, each of the two polypeptides in the antigen binding domain comprises a leader sequence for expression in the cell membrane.

The variable domain is connected to a constant domain. In particular, the C-terminal of the variable domain is connected to the N-terminal of the constant domain by a peptide bond.

In some embodiments, the antigen binding domain comprises two polypeptides, each of which comprises a variable and constant region derived from a TCR (e.g., tumor reactive TCR). In some embodiments, the two polypeptides in the antigen binding domain are connected by at least one disulfide bridge formed by cysteine residues in the constant domains. In some embodiments, the two polypeptides in the antigen binding domain are connected by more than one disulfide bridge formed by cysteine residues in the constant domains. In some embodiments, the two polypeptides in the antigen binding domain are connected by two disulfide bridges each formed by two cysteine residues in the constant domains.

In some exemplary embodiments, the TCR portion is derived from Radium 1 or DMF5 TCRs. In some embodiments, the antigen binding domain comprises or consists of SEQ ID NO:1 and SEQ ID NO:2, or sequences with at least 95% identity thereof. In some embodiments, the antigen binding domain comprises or consists of SEQ ID NO:1 and SEQ ID NO:16, or sequences with at least 95% identity thereof. In some embodiments, the antigen binding domain comprises or consists of SEQ ID NO:1 and SEQ ID NO:2 or 16, or sequences with at least 95% identity thereof, provided SEQ ID NO:1 comprises the three CDRs DSVNN, IPSGT and AVNAGNMLTF and provided SEQ ID NO:2 or 16 comprises the three CDRs MDHEN, SYDVKM and ASSSGVTGELFF. In some embodiments, the antigen binding domain comprises or consists of SEQ ID NO:8 and SEQ ID NO:9 or 17, or functional fragments thereof. In some embodiments, the antigen binding domain comprises or consists of SEQ ID NO:8 and SEQ ID NO: 9 or 17, or sequences with at least 95% identity thereof. In some embodiments, the antigen binding domain comprises or consists of SEQ ID NO:8 and SEQ ID NO:9 or 17, or sequences with at least 95% identity thereof, provided SEQ ID NO:8 comprises the three CDRs DRGSQS, IYSNGD and AVNFGGGKLIF and provided SEQ ID NO:9 or 17 comprises the three CDRs MRHNA, SNTAGT and ASSLSFGTEAFF.

As used herein, the constant domain is a fragment of an alpha chain, beta chain, gamma chain or delta chain which can be identified between the variable domain and the transmembrane domain of naturally occurring TCRs. As the terminology suggests, the sequences of these domains are quite conserved. In particular, the constant domains of human alpha (a) chain or beta 03) chains can be used. Examples of such constant domains can be represented by, for example, SEQ ID NO:3, SEQ ID NO:4, SEQ ID NO:10 or SEQ ID NO:11. It is preferred that the antigen binding domain comprises one constant domain from an alpha chain and one constant domain from a beta chain. The constant domain of human alpha chain usually comprises approximately 90 amino acid residues. The constant domain of human beta chain usually comprises approximately 130 amino acid residues. The constant domains may contain cysteine residues for connecting the two sequences in the antigen binding domain by one or more disulfide bridges.

In some exemplary embodiments, it was found that threonine in position 48 in the constant domain of alpha chains may be substituted by a cysteine residue. Such substitution is represented by the underlined C in SEQ ID NO:3 below:

PDPAVYQLRDSKSSDKSVCLFTDFDSQTNVSQSKDSDVYITDKCVLDMRS
MDFKSNSAVAWSNKSDFACANAFNNSIIPEDTFFPSPESSC

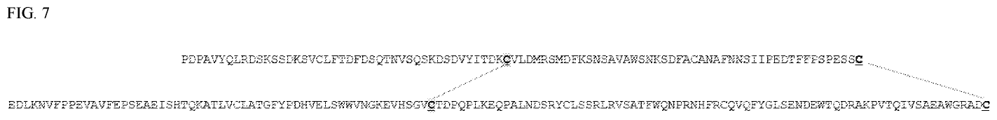

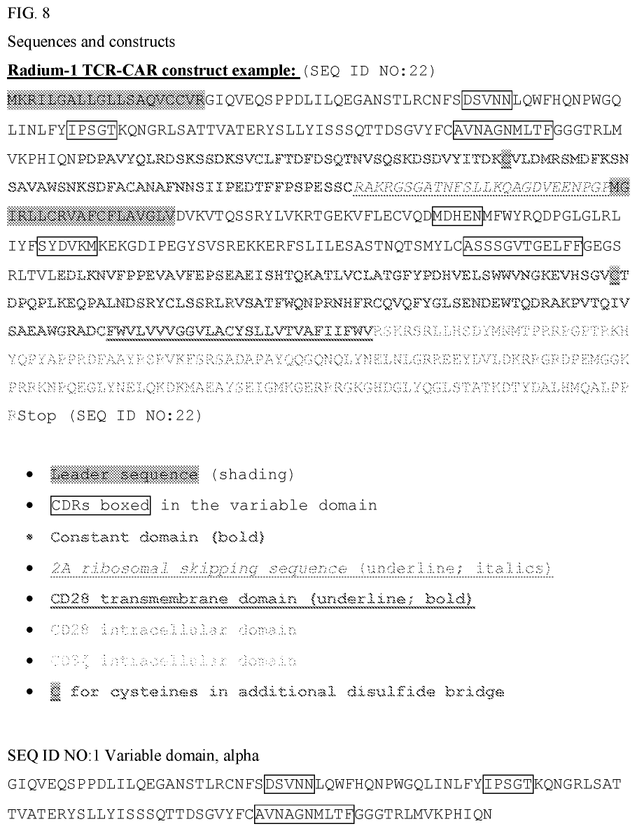

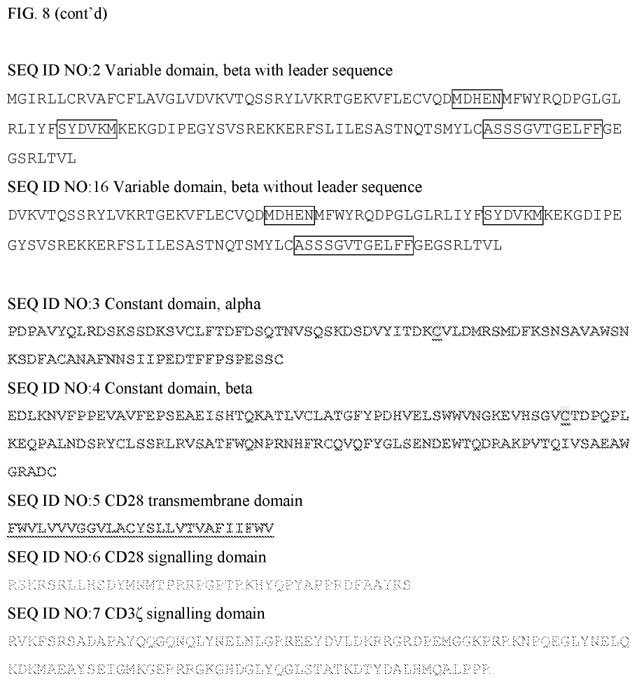

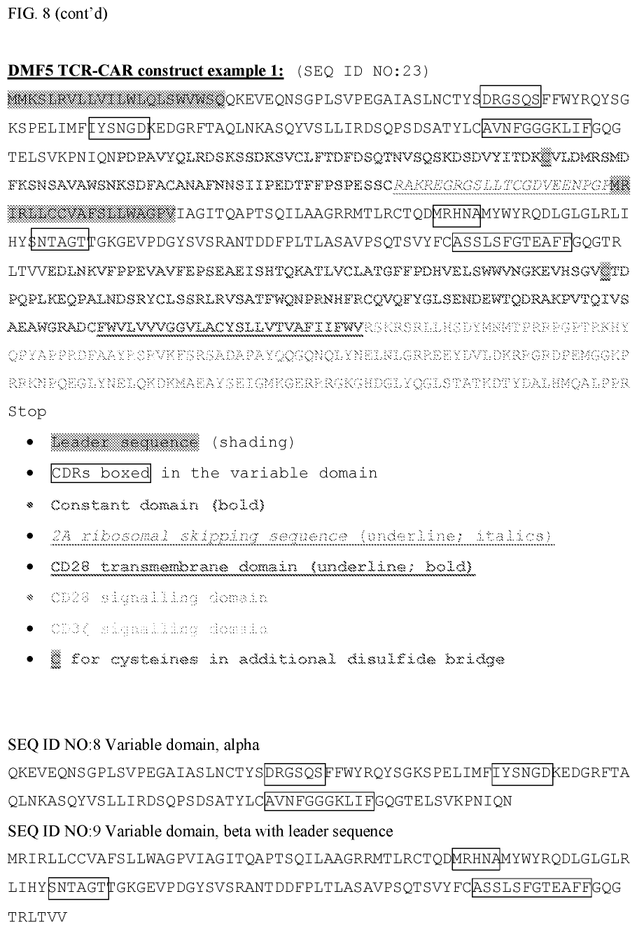

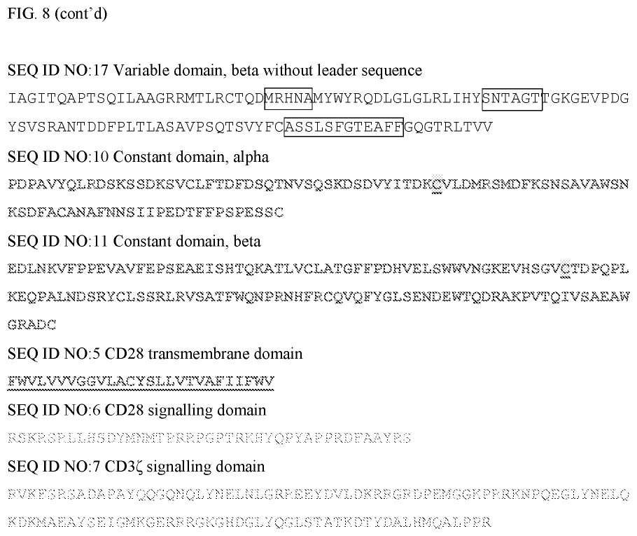

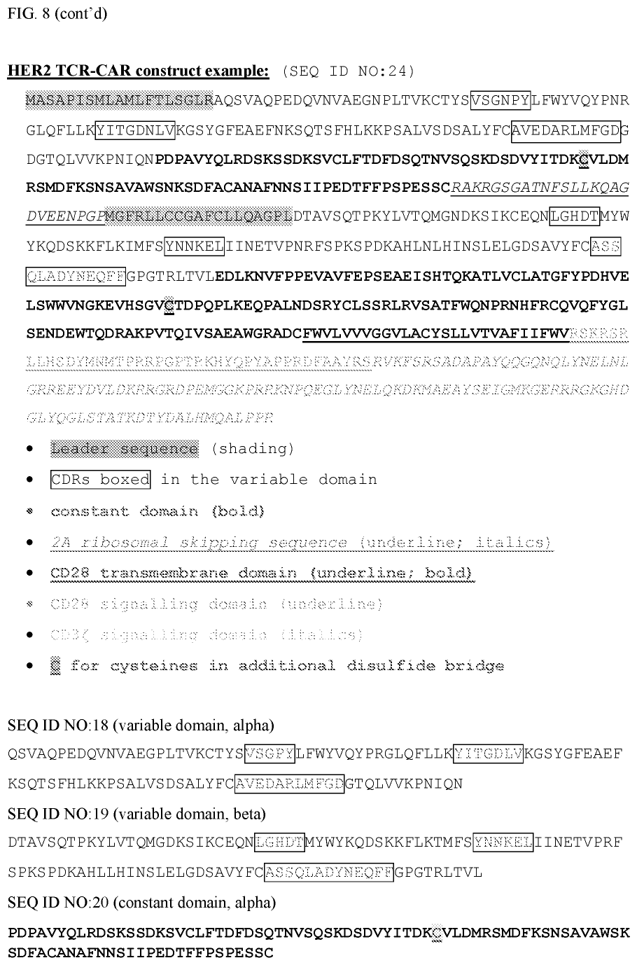

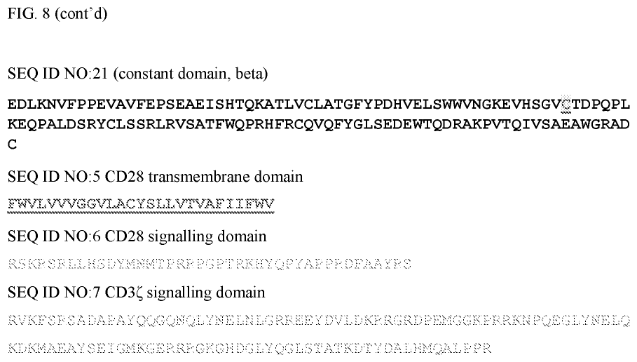

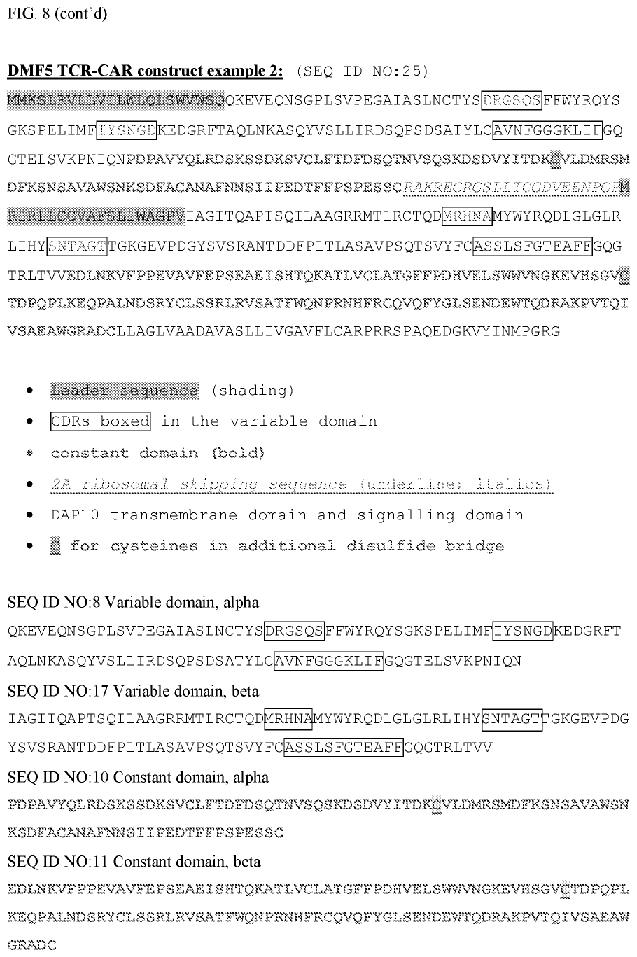

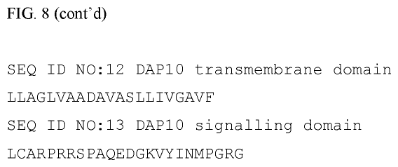

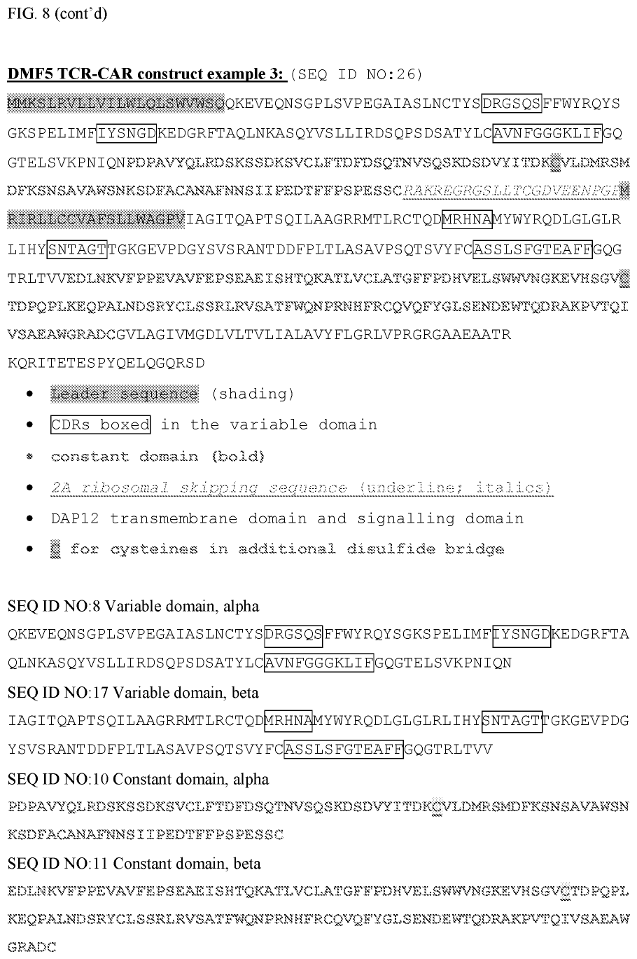

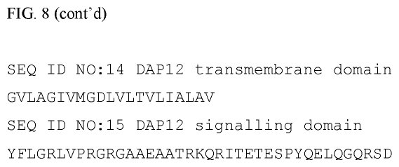

Accordingly, in some embodiments, the amino acid residues in position 48 and 91 of SEQ ID NO:3 are both cysteine residues. These cysteine residues may form interchain bridges to the constant domain of the beta chain (see FIG. 7).

In some exemplary embodiments, it was found that serine in position 57 in the constant domain of beta chains may be substituted by a cysteine residue. Such substitution is represented by the underlined C in SEQ ID NO:4 below:

EDLKNVFPPEVAVFEPSEAEISHTQKATLVCLATGFYPDHVELSWWVNGK
EVHSGVCTDPQPLKEQPALNDSRYCLSSRLRVSATFWQNPRNHFRCQVQF
YGLSENDEWTQDRAKPVTQIVSAEAWGRADC

Accordingly, in some embodiments, the amino acid residues in position 57 and 131 of SEQ ID NO:4 are both cysteine residues. These cysteine residues may form interchain bridges to the constant domain of the alpha chain (see FIG. 7).

Without being bound by theory, these substitutions mentioned above may allow the formation of an additional disulfide bridge between the constant domain from an alpha chain and the constant domain from a beta chain. These bridges may contribute to the stability of the antigen binding domain and may enable the TCR-CAR to efficiently transduce signals into immune effector cells

The present disclosure is not limited to particular transmembrane domains. The transmembrane domain may be derived either from a natural or from a synthetic source. Where the source is natural, the domain may be derived from any membrane-bound or transmembrane protein. Transmembrane regions of particular use in this invention may be derived from (i.e. comprise or consist of at least the transmembrane region(s) of) the alpha, beta or zeta chain of the T cell receptor, CD28, CD3 epsilon, CD45, CD4, CD5, CD8, CD8a, CD9, CD16, CD22, CD28, CD33, CD37, CD64, CD80, CD86, CD134, CD137, CD154, DAP10, or DAP12. Alternatively the transmembrane domain may be synthetic. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

For example, in some embodiments, the transmembrane domains from CD8a or CD28, or functional variants/fragments thereof may be used. In some embodiments, the transmembrane domain from CD28, represented by SEQ ID NO:5, is used. This transmembrane domain allows the TCR-CAR to bypass the endogenous TCR signalling machinery. In some embodiments, the transmembrane domain from the natural killer cell signaling adaptor molecule DNAX-activating protein 10 and 12 (DAP10 and DAP12) represented by SEQ ID NO:12 and SEQ ID NO:14 is used.

The present disclosure is not limited to particular intracellular signaling domains. Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Examples of signaling sequences that are of particular use in the invention include those derived from TCR zeta, FcR gamma, FcR beta, CD3 gamma, CD3 delta, CD3 epsilon, CD5, CD22, CD79a, CD79b, and CD66d. In some embodiments, the cytoplasmic domain comprises signalling domains from CD28, CD3, 4-1BB, OX40, ICOS etc. or functional variants/fragments of such domains. In some embodiments, a functional fragment of the CD28 signalling domain, represented by SEQ ID NO:6, is used. In some embodiments, a functional fragment of the CD28 signalling domain, represented by SEQ ID NO:6, is used together with the CD3 signalling domain represented by SEQ ID NO:7. In some embodiments, the intracellular signalling domains from DAP10 and DAP12, represented by SEQ ID NO:13 and SEQ ID NO:15 are used for signal transduction in natural killer cells.

In some embodiments, the TCR-CARs described herein are used in combination with a conventional CAR targeting surface epitopes via antigen binding domains from antibodies.

Embodiments of the present invention provide variants, fragments, etc. of the disclosed sequences. In some embodiments, substitutions are conservative or non-conservative substitutions. In some embodiments, variants and fragments retain the functionality of the original polypeptide. As described above, in some embodiments, variant are at least 90% (e.g., 90%, 91%, 92%, 93%, 94%, 95%, 95%, 97%, 98%, or 99%) identical to the original polypeptide.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277), preferably version 3.0.0 or later. The optional parameters 11644.000-EP7 used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows: (Identical Residues×100)/(Length of Alignment−Total Number of Gaps in Alignment).

In some embodiments, the transgene encoding for this construct is designed as a polycistronic gene and, in some embodiments, the TCR α and β coding sequences are linked by a 2A ribosomal skipping sequence, which ensures an equimolar production of the final heterodimer. The 2A sequence allows the release of the upstream protein and translation of the downstream gene. It is expected that the 2A also serves to bring the two subunits in contact, and improve their dimerization. Finally, the C-terminal part of each chain can be modified to carry additional tags/domains if needed. The extracellular domains can also be improved (addition of extra Cysteines) to increase the dimer stability.

The nucleic acid sequences coding for the desired molecules can be obtained using recombinant methods, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present invention also provides vectors in which a nucleic acid of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Any suitable method of introducing and expressing genes into a cell may be utilized. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

### II. Cells

In some embodiments, the present invention provides cells comprising the CAR described herein. The present invention is not limited to particular cell types. Examples include, but are not limited to, T cells, natural killer cells, NK-92 cells, and the like. Cells may be primary (e.g., from an autologous or heterologous donor) or immortalized cell lines.

In some embodiments, the present invention utilizes cells that are isolated from a subject and modified ex vivo. In such embodiments, prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Other washing methods include a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, or from samples where there are many tumor cells present (i.e., leukemic blood, tumor tissue, etc.). Such populations of cells may have therapeutic value and would be desirable to obtain.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rittman.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express a desirable TCR-CAR of the invention, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (T_{C}, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{c} cells. Accordingly, depending on the purpose of treatment, infusing a subject with a T cell population comprising predominately of T_{H} cells may be advantageous. Similarly, if an antigen-specific subset of T_{c} cells has been isolated it may be beneficial to expand this subset to a greater degree.

### III. Therapeutic Application

The CARs described herein find use in a variety of therapeutic application. In some embodiments, cells expressing TCR-CARs described herein are used in anti-cancer adoptive cell transfer (ACT). In some embodiments, the cells are injected into patients whose tumour is positive for the targeted epitope (peptide) and HLA allele. In some embodiments, this redirects effector cells with a killing capacity such as NK cells (and any non T cell) against a T cell target and also other type of immune effector cells (macrophage) with regulatory function such as cytokine release or antigen presentation.

Experiments conducted during the course of development of embodiments of the present invention demonstrated TCR-CARs directed at peptides of malignant melanoma and colorectal cancer, although the present invention is not limited to a particular cancer.

In one embodiment, the present invention includes a type of cellular therapy where cells are genetically modified to express a CAR and the CAR T cell is infused to a recipient in need thereof. The infused cell is able to kill tumor cells in the recipient. Unlike antibody therapies, CAR cells are able to replicate in vivo resulting in long-term persistence that can lead to sustained tumor control.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

The CAR-modified cells of the invention also find use in ex vivo methods of inducing a cancer-specific immune response in a mammal. Preferably, the mammal is a human. With respect to ex vivo therapy, at least one of the following occurs in vitro prior to administering the cell into a mammal: i) expansion of the cells,

ii) introducing a nucleic acid encoding a CAR to the cells, and/or iii) cryopreservation of the cells.

Any suitable ex vivo procedure may be utilized. In one exemplary method, cells are isolated from a mammal (preferably a human) and genetically modified (e.g., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

A procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

The CAR-modified cells of the present invention may be administered either alone, or as a pharmaceutical composition in combination with diluents and/or with other components such as IL-2 or other cytokines or cell populations. Briefly, pharmaceutical compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the T cell compositions of the present invention are administered to a patient by intradermal or subcutaneous injection. In another embodiment, the T cell compositions of the present invention are preferably administered by i.v. injection. The compositions of T cells may be injected directly into a tumor, lymph node, or site of infection.

In certain embodiments of the present invention, cells describe hereinare administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rittman. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

### EXPERIMENTAL

### Example 1

### Methods

Cell lines, Media, Chemicals and Peptides.

T cells were obtained from buffy coats from healthy blood donors from the blood bank (Ullevål hospital, Oslo, Norway). J7631 (obtained from M. Heemskerk, Leiden University Medical Center, The Nederlands) were maintained in RPMI (PAA, Paschung, Austria) supplemented with 10% HyClone FCS (HyClone, Logan, Utah, USA) and gentamicin (50 μg/mL) K562 (ATCC, CCL-243), Granta-519 (DSMZ, ACC 342) and T2 cells were maintained in the same medium. The packaging cells were the modified Human Embryonic Kidney cells-293, Hek-Phoenix (Hek-P) and they were grown in DMEM (PAA) with 10% FCS. T cells were grown in CellGro DC medium (CellGenix, Freiburg, Germany) supplemented with 5% heat-inactivated human serum (Trina Bioreactives AG, Nänikon, Switzerland), 1.25 mg/mL N-acetylcysteine (Mucomyst 200 mg/mL, AstraZeneca AS, London, UK), 0.01 M HEPES (Life Technologies, Norway) gentamycin 0.05 mg/mL (Garamycin, Schering-Plough Europe, Belgium). NK-92 cells were cultured and maintained in X-Vivo 10 medium supplemented with 5% heat-inactivated HS and 500 IU/mL IL-2. The TGFbR2 frameshift peptide131-139, RLSSCVPVA was provided by Norsk Hydro ASA, (Porsgrunn, Norway). The MART-1 peptide26-35 EAAGIGILTV was manufactured by Prolmmune Ltd (Oxford, UK) and MART-1 dextramer was from Immudex (Copenhagen, Denmark). DNA Constructs. The transmembrane and cytosolic domain from the CAR25 domain was added on to the previously described soluble TCR24 by overlapping PCR by using the following primers: CAR template (5′-3′)

forward
(SEQ ID NO: 27)
ggtagagcagactgtggtaaattttgggtgctggtggtgg (1),
reverse
(SEQ ID NO: 28)
ctcgagttagcgaggaggcagggcctgcatgtgaag (2),
sTCR template forward (Radium1)
(SEQ ID NO: 29)
caccatgaagaggatat (3),
(DMF5)
(SEQ ID NO: 30)
caccatgatgaaatcct (4),
reverse
(SEQ ID NO: 31)
ccaccaccagcacccaaaatttaccacagtctgctctaccc (5).

The two PCR products were subsequently combined into the TCR-CAR using the following primer pair (3) and (2) for Radium-1 and (4) and 3(2) for DMF5. The final PCR product was cloned into pENTR (Themofisher, Waltham, Mass., USA). Sequence verified constructs were recombined into a Gateway-modified pMP71 (retroviral vector) or pCIpA102 (mRNA synthesis construct) as described (Walchli, S. et al. A practical approach to T-cell receptor cloning and expression. PLoS One 6, e27930 (2011)). TCR expression constructs used here were described for DMF5 and Radium-1, respectively (Inderberg, E. M. et al. T cell therapy targeting a public neoantigen in microsatellite instable colon cancer reduces in vivo tumor growth. Oncoimmunology 6, (2017); Walchli, S. et al. A practical approach to T-cell receptor cloning and expression. PLoS One 6, e27930 (2011)). The HLA-A2 construct was previously described (Walchli, S. et al. Invariant chain as a vehicle to load antigenic peptides on human MHC class I for cytotoxic T-cell activation. Euro. J Immunol. 44, 774-784, (2014)), Addgene (Plasmid #85162). In vitro mRNA transcription. The in vitro mRNA synthesis was performed essentially as previously described (Aggen, D. H. et al. Identification and engineering of human variable regions that allow expression of stable single-chain T cell receptors. Protein Eng Des Sel 24, 361-372 (2010)). Anti-Reverse Cap Analog (Trilink Biotechnologies Inc., San Diego, Calif., USA) were used to cap the RNA. The mRNA quality was assessed by agarose gel electrophoresis and Nanodrop (Thermo Fisher Scientific).

In Vitro Expansion and Electroporation of T Cells.

T cells from healthy donors were expanded using a protocol adapted for GMP production of T cells employing Dynabeads CD3/CD28 as described (Almasbak, H. et al. Transiently redirected T cells for adoptive transfer. Cytotherapy 13, 629-640, (2011)). In brief, PBMCs were isolated from buffy coats by density gradient centrifugation and cultured with Dynabeads (Dynabeads® ClinExVivo™ CD3/CD28, ThermoFischer, Oslo, Norway) at a 3:1 ratio in complete CellGro DC Medium with 100 U/mL recombinant human interleukin-2 (IL-2) (Proleukin, Prometheus Laboratories Inc., San Diego, Calif., USA) for 10 days. The cells were frozen and aliquots were thawed and rested in complete medium before transfection. Expanded T cells were washed twice and resuspended in CellGro DC medium (CellGenix GmbH) to 70×10⁶ cells/mL. The mRNA was mixed with the cell suspension at 100 μg/mL, and electroporated in a 4-mm gap cuvette at 500 V and 2 ms using a BTX 830 Square Wave Electroporator (BTX Technologies Inc., Hawthorne, N.Y., USA). Immediately after transfection, T cells were transferred to complete culture medium at 37° C. in 5% CO₂ overnight.

Retroviral Transduction of NK-92 and Preparation of K562 (HLA-A2).

Viral particles were produced as described (Walchli et al, supra) and were used to transduce NK-92 and K562 cells as follows: Spinoculation was performed with 1 Volume of retroviral supernatant mixed with 1 Volume of cells (0.3 M/mL) in a 12-well (2 mL final) or a 24-Well (1 mL final) non-treated plate (Nunc A/S, Roskilde, Denmark) pre-coated with retronectin (50 μg/mL, Takara Bio. Inc., Shiga, Japan). NK-92 cells were spinoculated twice at 32° C. at 750×g for 60 min. Cells were then harvested with PBS-EDTA (0.5 mM) and grown in their regular medium.

Functional Assay and Flow Cytometry.

K562 (HLA-A2) or Granta-519 cells were loaded with peptide overnight at 37° C. in a 5% CO2 incubator. Effector cells were stimulated with target cells at an effector-to-target (E:T) ratio of 1:2 for 5 hours at the same conditions as above. Conjugated CD107a was added to the cells prior to incubation. Irrelevant or no peptide served as a negative control. The following antibodies were used: Vβ3-FITC (Beckman Coulter-Immunotech SAS, France), CD3-eFluor450, CD56-eFluor, CD107a-PE-Cy5, TNFα-PE (BD Biosciences, USA), IL2-APC, IFNγ-FITC (eBiosciences, ThermoFischer). Cells were washed in flow buffer (FB, phosphate buffered saline (PBS) with 2% human bovine serum albumin (BSA) and 0.5 μM EDTA). For dextramer and antibody staining, cells were incubated for 30 minutes at room temperature (RT) with the recommended dilution in FB. If fixed, cells were incubated in FB containing 1% paraformaldehyde. For intracellular staining Perm/Wash Buffer was used (BD Biosciences) according to manufacturer's protocol. All antibodies were purchased from eBioscience, USA, except where noted. Cells were acquired on a BD FACSCanto II flow cytometer and the data analyzed using FlowJo software (Treestar Inc., Ashland, Oreg., USA). Plotting and statistical analysis were performed using GraphPad prism software (La Jolla, Calif., USA).

Bioluminescence-Based Cytotoxicity Assay.

Luciferase-expressing tumor cells were counted and resuspended at a concentration of 3×105 cells/mL. Xenolight D-Luciferin potassium salt (75 μg/mL; Perkin Elmer, Oslo, Norway) was added to tumor cells which were placed in 96-well white round bottomed plates at 100 μL cell suspension/well in triplicates. Subsequently, effector cells were added as indicated effector-to-target (E:T) ratios. In order to determine baseline cell death and maximal killing capacity, three wells were left with only target cells and another three with target cells in 1% Triton™ X-100 (Sigma-Aldrich). Cells incubated at 37° C. for 2 hours.

Bioluminescence (BLI) was measured with a luminometer (VICTOR Multilabel Plate Reader, Perkin Elmer) as relative light units (RLU). Target cells that were incubated without any effector cells were used to determine baseline spontaneous death RLU in each time point. Triplicate wells were averaged and lysis percentage was calculated using following equation: % specific lysis=100×(spontaneous cell death RLU−sample RLU)/(spontaneous death RLU−maximal killing RLU). Plotting and statistical analysis were performed using GraphPad prism software.

Cytokine Measurements.

Cytokines released from transduced or non-transduced NK-92 cells incubated with Granta-519 cells were collected after 24 hours of co-culture. Cytokines in supernatants were measured by using the Bio-Plex Pro™ Human Cytokine 17-plex Assay (Bio-Rad Laboratories, Hercules, Calif., USA) according to manufacturer's protocol on a Bio-Rad Bio-Plex 100 system. Plotting and statistical analyses were performed using GraphPad prism software.

### Results

Design of TCR-CAR.

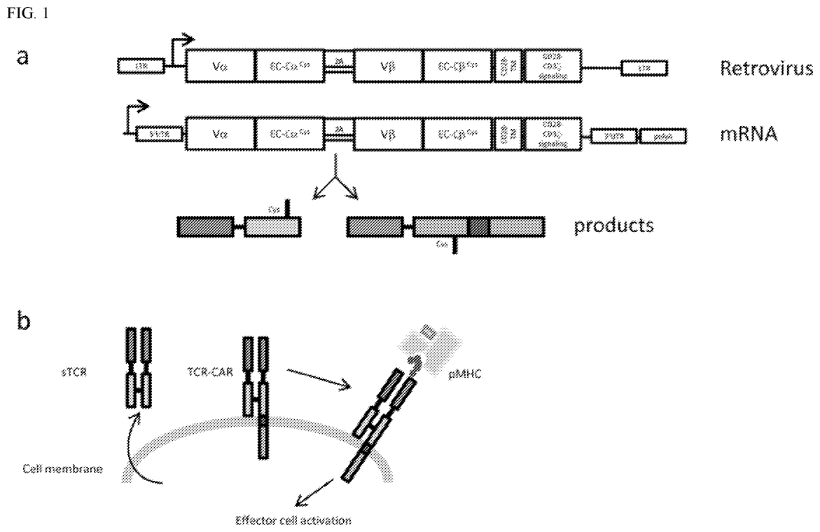

It was previously shown that one could efficiently express soluble TCR (sTCR) in Human embrionic kidney (Hek) cells and derivative (such as Hek-Phoenix) (Walseng, E. et al. Soluble T-cell receptors produced in human cells for targeted delivery. PLoS One 10, (2015)). High yields (4 mg/L) of active material were obtained by taking advantage of the 2A-based expression system (Kim, J. H. et al. High cleavage efficiency of a 2A peptide derived from porcine teschovirus-1 in human cell lines, zebrafish and mice. PLoS One 6, e18556, (2011)). These results were per se not predictable as the release of the two separated soluble chains would not mechanically result in the formation of a stable molecule, as a likely outcome could be their degradation in the ER or never be sent to the plasma membrane. Since synthesis and export of sTCR generation were possible, a related construct in which the TCRβ chain was fused to an artificial signalling domain similar to the one used for CARs was designed (FIG. 1a): namely CD28 transmembrane coding sequence followed by two signalling modules (CD28 and CD3ζ). In addition, a cysteine replacement was performed on the constant domain (C-domain) in order to increase the TCR dimer stability (Cohen, C. J. et al. Enhanced antitumor activity of T cells engineered to express T-cell receptors with a second disulfide bond. Cancer Res 67, 3898-3903 (2007); Walseng et al., supra). This TCR-CAR cassette was subcloned into two different expression systems, namely MP71 retroviral vector and mRNA synthesis vector using the strategy published earlier (Walchli et al., supra).

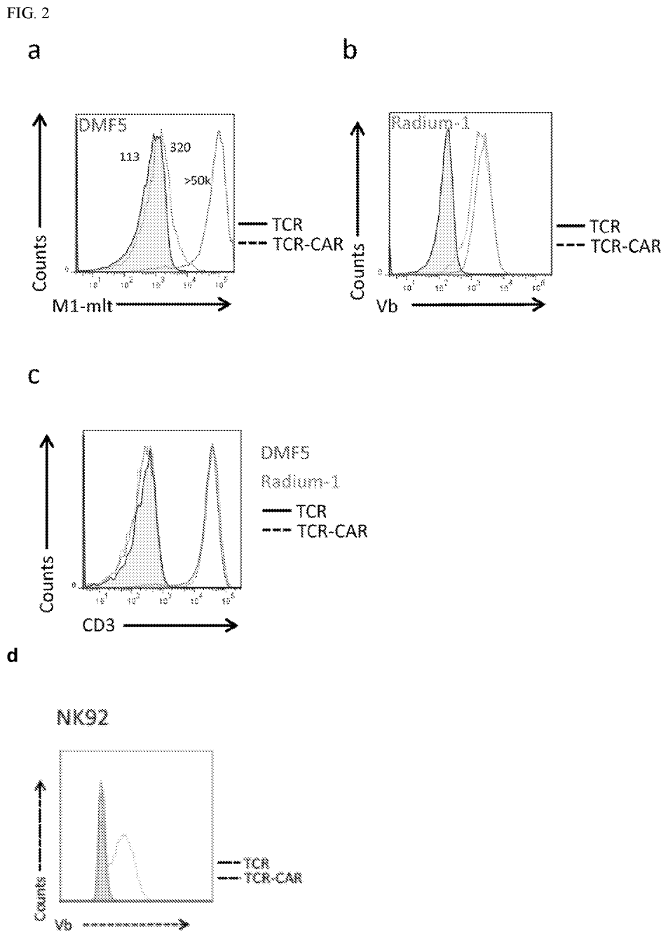

It was expected that the protein product of the TCR-CAR construct would be exported to the plasma membrane like a receptor and that upon pMHC encounter, it would bind to its substrate and signal (FIG. 1b). Two MHC-Class I restricted TCRs, DMF5 (Johnson, L. A. et al. Gene transfer of tumor-reactive TCR confers both high avidity and tumor reactivity to nonreactive peripheral blood mononuclear cells and tumor-infiltrating lymphocytes. J. Immunol. 177, 6548-6559 (2006)) and Radium-1 (Inderberg, E. M. et al. T cell therapy targeting a public neoantigen in microsatellite instable colon cancer reduces in vivo tumor growth. Oncoimmunology 6, e1302631, doi:10.1080/2162402X.2017.1302631 (2017)), which are directed against the MART-1 peptide (Johnson et al. supra; Inderberg et al. supra; Kim et al., supra; Walchli et al, supra; Saeterdal, I. et al. Frameshift-mutation-derived peptides as tumor-specific antigens in inherited and spontaneous colorectal cancer. Proc. Nat'l Acad. Sci. 98, 13255-13260, (2001); Heemskerk, M. H. et al. Redirection of antileukemic reactivity of peripheral T lymphocytes using gene transfer of minor histocompatibility antigen HA-2-specific T-cell receptor complexes expressing a conserved alpha joining region. Blood 102, 3530-3540 (2003); Birnbaum, M. E. et al. Molecular architecture of the alphabeta T cell receptor-CD3 complex. Proc. Nat'l Acad. Sci. 111, 17576-17581, (2014); Krshnan, L., et al., A conserved alphabeta transmembrane interface forms the core of a compact T-cell receptor-CD3 structure within the membrane. Proc. Nat'l Acad. Sci. 113, E6649-E6658, (2016); Almasbak, H. et al. Transiently redirected T cells for adoptive transfer. Cytotherapy 13, 629-640, (2011); Daniel-Meshulam, I., et al., Front. Immunol. 3, 186, (2012) (EAAGIGILTV) and TGFbR2 frameshift neoantigen peptide131-139 (RLSSCVPVA) (Saeterdal et al., supra), respectively were selected. It was first tested whether these constructs could be efficiently produced and sent to the plasma membrane. Expression of TCR-CAR was compared with their corresponding full-length TCR in J76 cells, which are TCR negative but become CD3 positive upon TCR expression (Heemskerk et al, supra). DMF5 TCR and TCR-CAR were detected using a commercially available MART-1 dextramer (FIG. 2a). A weak expression of DMF5 TCR-CAR was detected, indicating that it was either not well exported to the membrane or the protein was not stable when expressed in this format. Since there was no multimer available for Radium-1 TCR staining, an antibody against the Vbeta-chain of Radium-1 (anti-Vb3, Vb) was used to detect both constructs (FIG. 2b). Unlike what was observed with DMF5, Radium-1 TCR-CAR was expressed with a similar efficiency as its full-length TCR counterpart. On the other hand, DMF5 showed limited ability to bind the dextramer. Since the TCR-CAR proteins were expressed at the plasma membrane (FIGS. 2a and b), we tested their ability to recruit CD3.

When a TCR is expressed in J76 cells, they become CD3 positive. However, CD3 staining showed that TCR-CAR did not interact with endogenous CD3 since J76 remained CD3 negative (FIG. 2c). This is in line with recent reports proposing an interaction between CD3 and TCR through the native transmembrane domains (Birnbaum et al., supra; Krshnan et al., supra), which is not necessarily present in the TCR-CARs herein and thus indicates that TCR-CAR may acts independently of endogenous TCR signaling machinery, possibly due to the presence of CD28 transmembrane domain. As for classical CAR constructs, it was contemplated that predicted that the construct would bypass the “CD3-block” due to the presence of CD28 transmembrane domain.

It was also expected that TCR-CARs lacking the native TCR transmembrane domains would not compete for the endogenous CD3. Radium-1 TCR and TCR-CAR were expressed at similar levels as detected by the specific Vb antibody. This indicates that Radium-1 TCR-CAR was well folded and comprised a Vb and a Va chains. On the other hand, DMF5 TCR-CAR was less efficiently produced. This was surprising as this TCR was very stable when prepared as sTCR23. It was observed that even sorted cells had a tendency to lose the MART-1-dextramer positive signal after several passages, indicating that DMF5 TCR-CAR may become detrimental to the cells expressing it at high levels. Dextramer staining is a more stringent measure of expression than Vb staining since dextramer will only detect correctly folded and heterodimerized TCR chains. In conclusion, it was observed that both TCR-CAR constructs were expressed at the membrane in J76 cells, but the level was lower than the full-length TCR. This could be due to a poor stability of the TCR-CAR construct. However, the lack of CD3 dependency represents a great advantage over classical overexpression of full-length TCR as it also means that TCR-CAR expression can be extended to other cells than T cells.

T Cells Redirected by TCR-CAR.

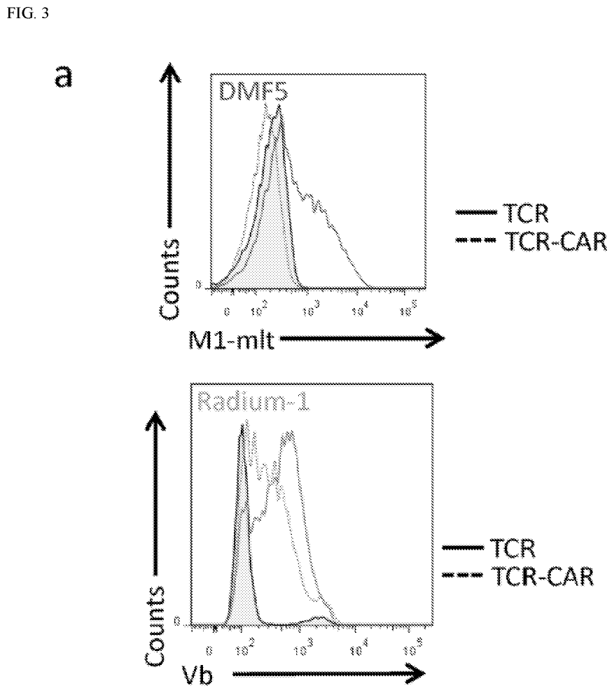

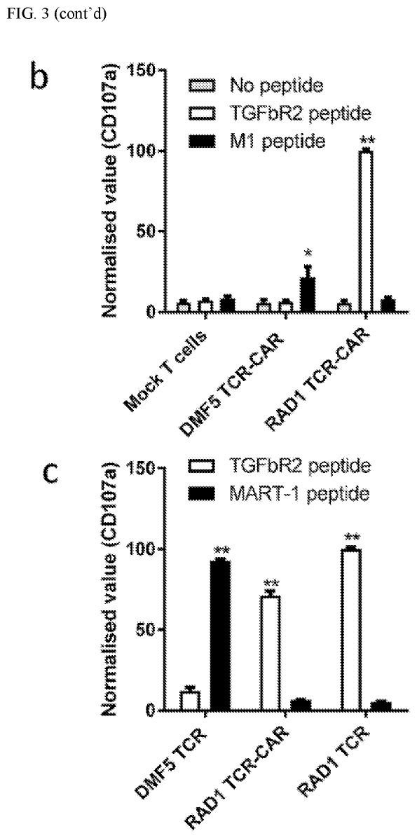

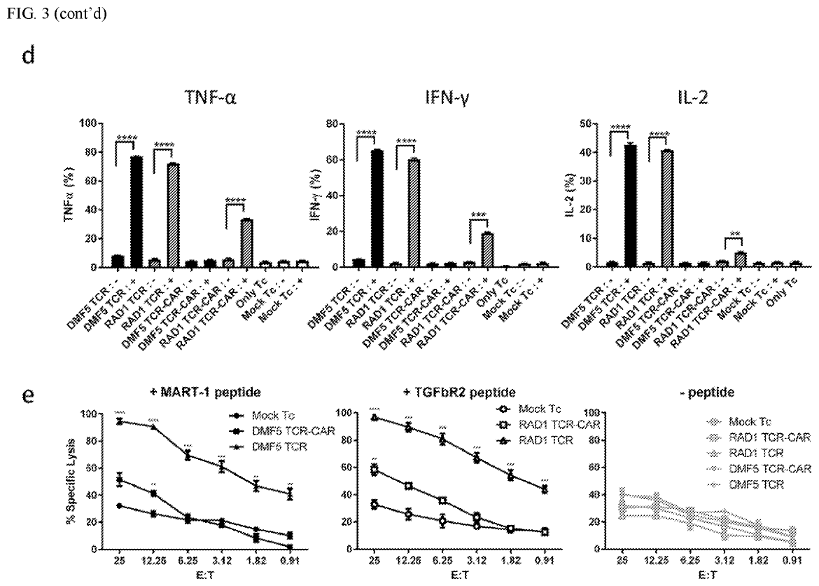

The activity of a TCR can be evaluated in a functional assay in which target cells positive for the specific MHC loaded with the relevant peptide are used. TCR-CAR was introduced into primary T cells isolated from PBMC by mRNA electroporation (Krshnan et al., supra) and the protein expression analyzed by flow cytometry (FIG. 3a). As shown both full-length TCRs were well expressed, TCR-CAR was detected at a lower level than Radium-1 TCR, and DMF5 TCR-CAR were not detected by multimer. Since multimer staining is not a highly sensitive method, the fact that DMF5 TCR-CAR was not detected by multimer does not mean that the protein was not present.

It was therefore tested whether primary T cells could be redirected against specific targets. Both the TCRs used here being HLA-A2 restricted, a myelogenous leukaemia cell line, K562, was transduced with HLA-A2 and used as APC. These cells pre-loaded with the indicated peptides were incubated with TCR-CAR redirected T cells. The T-cell activation was monitored five hours later by detecting the presence of the degranulation marker CD107a at the plasma membrane of the T cells. As shown (FIG. 3b), only the correct combination of pMHC was recognized by TCR-CAR. Mock electroporated T cells were used as a negative control and showed no stimulation. When DMF5 TCR-CAR was electroporated, a slight but significant activation was observed (FIG. 3b). This indicates that although DMF5 TCR-CAR expression in T cells was not detectable, some TCR-CAR activity was still monitored. This is in agreement with previous observations using mRNA electroporated conventional CAR T cells showing that even at protein levels not detectable by specific anti-CAR antibodies, the activity was present (Almasbak et al., supra). On the other hand, Radium-1 TCR-CAR showed a sustained pMHC-specific stimulation, which matched the expression of the TCR-CAR detected by Vb3 staining (FIG. 3a). In order to study the level of stimulation TCR-CAR could induce, the experiment comparing Radium-1 TCR-CAR with full-length Radium-1 TCR was repeated and it was demonstrated that both constructs had the capacity to trigger degranulation (FIG. 3c).

Finally, the ability of the constructs to redirect T cells and trigger cytokine release and target cell killing was assayed. In agreement with the CD107a expression, DMF5 TCR-CAR did not trigger cytokine release, but did significantly kill peptide loaded APC (FIGS. 3d and e, respectively). On the other hand, Radium-1 TCR-CAR redirected T cells were able to produce cytokines in a peptide-dependent manner and significantly kill target cells loaded with specific peptides (FIGS. 3d and e). Radium-1 TCR performed more efficiently than TCR-CAR in both assays, but the TCR-CAR construct was functional, reaching statistical significance, indicating that the receptor was potent. Taken together, the data show that when the TCR-CAR construct was expressed in primary T cells: (1) the recognition part of TCR-CAR maintained its specificity when fused to an artificial signalling domain and (2) the signalling part when fused to TCR could recruit endogenous signalling components to trigger degranulation and target cell killing. Although the values for both TCR-CARs were lower than the ones obtained with the full-length constructs, TCR-CARs were functional. This is largely explained by the difference in expression between full-length TCR and TCR-CAR, but may also be influenced by other mechanisms such as non-optimal signalling for T cells when using target recognition domains from TCR rather than antibodies. This is important because TCR-CAR design can be improved: antibody-based CARs have high affinity for their target, and tandem CD28-CD3 signalling modules may be sufficient for high affinity binding.

Compared to CAR, TCR binding to pMHC is considered to be of relatively low affinity and it may be helpful to increase the number or the potency of the signalling boxes in the TCR-CAR construct in order to optimize the cytokine release and killing efficiency. TCR redirection of patient T cells can be improved by different means (Daniel-Meshulam et al, supra), but influencing the signalling has rarely been exploited (Palmer, D. C. et al. Cish actively silences TCR signalling in CD8+ T cells to maintain tumor tolerance. J. Exper. Med. 212, 2095-2113, (2015)). Indeed, it was previously reported that CD3 overexpression could improve TCR redirection potency (Ahmadi, M. et al. CD3 limits the efficacy of TCR gene therapy in vivo. Blood 118, 3528-3537, (2011)). This improvement may result from the increased number of TCR molecules at the plasma membrane, including the endogenous TCR, which could result in increased mispairing, hence off-target effects. TCR-CAR did not compete for CD3 and signalled without being affected by the presence of endogenous TCRs. T cell like redirection of NK cells.

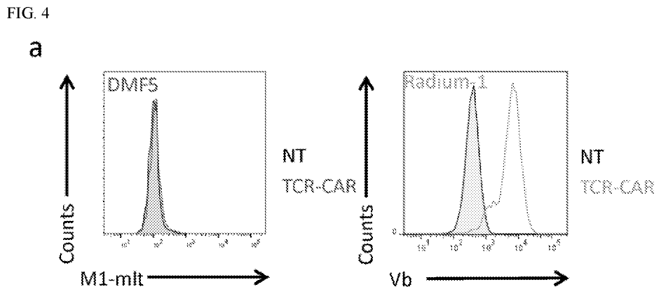

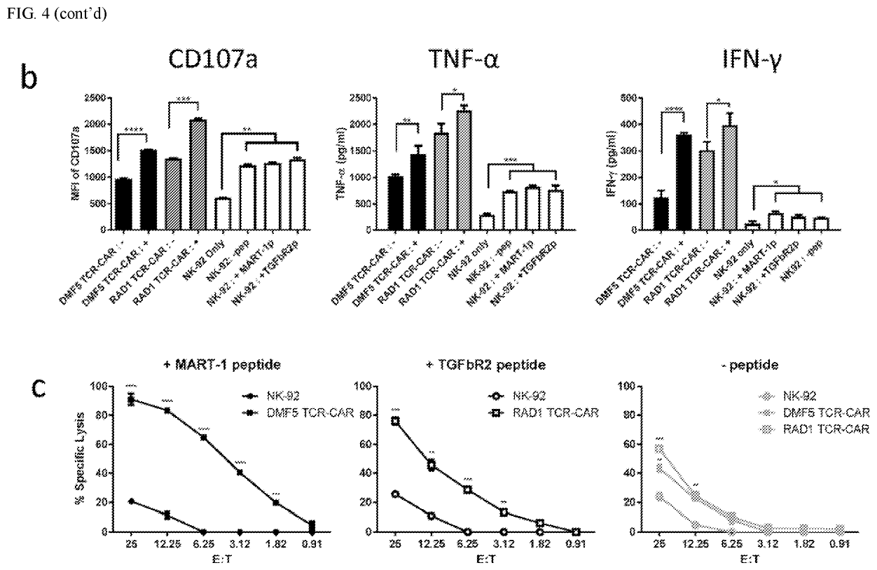

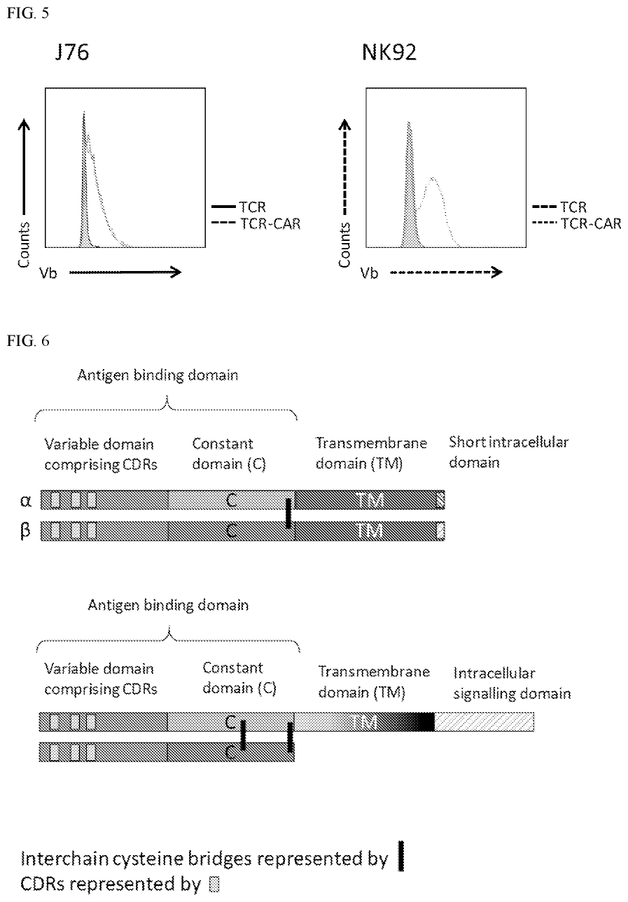

TCR-CAR carrying its own signalling units may redirect other killer cells than T cells. This was tested this by redirecting the non-T cell line, NK-92 which is a clinically approved natural killer cell line (Klingemann, H., et al., Natural Killer Cells for Immunotherapy—Advantages of the NK-92 Cell Line over Blood NK Cells. Front. Immunol. 7, 91, (2016); Suck, G. et al. NK-92: an ‘off-the-shelf therapeutic’ for adoptive natural killer cell-based cancer immunotherapy. Cancer Immunol. Immunother.: CII 65, 485-492, (2016)). It was first confirmed that NK-92 cells were not able to express a full-length TCR by electroporating them with mRNA encoding either Radium-1 TCR or Radium-1 TCR-CAR and staining them with an anti-Vb3 antibody (FIG. 5). As shown, only the TCR-CAR construct was detected at the cell surface of NK-92, whereas in the same conditions the T-cell line J76 expressed bothconstructs. Therefore, NK-92 cells were not able to express a full-length TCR at their cell surface. NK-92 cells were retrovirally transduced with the TCR-CAR constructs and after two rounds of spinoculation a large population of Vb3-positive NK-92 cells was obtained, indicating that the TCR-CAR could stably be expressed, folded and targeted at the surface of a non-T cell line (FIG. 4a). In contrast, the DMF5 TCR-CAR was not detected using multimer (FIG. 4a). Functional assays were then performed in order to study the activity of TCR-CAR in a non-T cell effector cell line, NK-92. To this end, the target cells were changed since K562 are commonly used as NK cell targets, and may generate elevated background responses, thus reducing the impact of the TCR stimulation.

In a functional assay to detect CD107a, it was observed that CD107a signal was high in the presence of different target cells. This may be due to NK-92 natural reactivity against tumour cell lines. Different HLA-A2 positive cell lines were tested and the “most resistant” to NK-92 in a killing assay and the B cell lymphoma cell line Granta-519, an HLA-A2 positive transformed mantle cell lymphoma, showed the lowest reactivity. They were co-incubated with NK-92-TCR-CAR after loading or not with the relevant peptide and cytokine release and killing activity of redirected NK-92 cells was assayed (FIGS. 4b and c, respectively). The degranulation marker CD107a expression and different cytokines (IFN-γ, TNF-α) upon target stimulation were assayed. As shown, NK-92 incubated with Granta-519 was stimulated (FIG. 4b, white columns) compared with NK-92 alone. However, the stimulation was significantly increased when TCR-CARs were expressed in NK-92 in the presence of peptide-loaded targets. Thus both TCR-CARs were expressed in NK-92 cells, and even if not detectable, were able to trigger pMHC-specific cytokine release. The overall background was higher in TCR-CAR expressing cells, indicating that the constructs were functional and gave some activation of NK-92 cells without binding their target. The capacity of NK-92 and NK-92-TCR-CAR cells to kill target cells (FIG. 4c) was next tested. The enhanced killing of peptide loaded cells was observed even at low E:T ratio, indicating that the killing was sensitive. In addition, at high E:T ratio NK-92 cells could kill target cells independently of the pMHC presence (FIG. 4c circles, maximum killing in the three conditions is 30% at E:T 1:25), TCR-CAR expression dramatically improved the recognition and the killing of the targets. Although not detectable by multimer staining, DMF5 TCR-CAR modified NK-92 cells became much more potent killers of MART-1 peptide loaded tumour cells than non-modified NK-92 cells, indicating that this TCR-CAR, even at low expression, was active and specific.

Killing was performed using unloaded Granta-519 as targets (FIG. 4c, right panel). This showed that despite specific TCR-dependent killing in the presence of peptide, killing of non loaded targets was observed to a higher degree and in an E:T ratio dependent manner by the TCR-CAR expressing NK-92 cells compared to NK-92 cells. This is in agreement with the increased basal cytokine release in NK-92 cells expressing TCR-CAR (FIG. 4b, black and grey columns) and demonstrates that the presence of TCR-CAR activated NK-92 cells.

In conclusion, TCR-CARs were able to redirect cells other than T cells to generate a TCR-dependent killing. Collectively, these data show that TCR-CAR expands the TCR expression spectrum to cells other than T cells. NK-92 cells have previously been exploited either naked or redirected with CAR. Tumour-specific surface antigen targets being scarce, TCR-CAR redirection opens new opportunities for targeting of NK cell-based adoptive transfer.

1. A chimeric antigen receptor (CAR), comprising:
a) an extracellular antigen binding domain that specifically binds to HLA complexes presenting peptides, wherein said extracellular antigen binding domain comprises two polypeptides, wherein each of said polypeptides comprises a variable domain and a constant domain;
b) a single transmembrane domain operably linked to said extracellular antigen binding domain; and
c) an intracellular signaling domain operably linked to said transmembrane domain.

2. The receptor of claim 1, wherein said two polypeptides are connected by at least two disulfide bridges, each bridge formed by two cysteine residues in the constant domains of said polypeptides.

3. The receptor of claim 1, wherein the transmembrane domain comprises
a) SEQ ID NO:5 or sequences at least 90% identical to SEQ ID NO:5;
b) SEQ ID NO:12 or sequences at least 90% identical to SEQ ID NO:12; or
c) SEQ ID NO:14 or sequences at least 90% identical to SEQ ID NO:14.

4. The receptor of claim 1, wherein the intracellular signaling domain comprises:
a) SEQ ID NO:6 or sequences at least 90% identical to SEQ ID NO:6;
b) SEQ ID NO:13 or sequences at least 90% identical to SEQ ID NO:13; or
c) SEQ ID NO:15 or sequences at least 90% identical to SEQ ID NO:15.

5. The receptor of claim 1, wherein the intracellular signaling domain comprises SEQ ID NO:6 and SEQ ID NO:7.

6. The receptor of claim 1, wherein the antigen binding domain comprises SEQ ID NO:1 and SEQ ID NO:2, or functional fragments thereof.

7. The receptor of claim 1, wherein the antigen binding domain is derived from a tumor reactive T cell receptor.

8. The receptor of claim 7, wherein the antigen binding domain comprises SEQ ID NO:1 and SEQ ID NO:2, or sequences with at least 95% identity to SEQ ID NOs: 1 and 2.

9. The receptor of claim 8, wherein the antigen binding domain comprises SEQ ID NO:1 and SEQ ID NO:2, or sequences with at least 95% identity to SEQ ID NOs: 1 and 2, provided SEQ ID NO:1 comprises the three CDRs DSVNN, IPSGT and AVNAGNMLTF and provided SEQ ID NO:2 or 16 comprises the three CDRs MDHEN, SYDVKM and ASSSGVTGELFF.

10. The receptor of claim 7, wherein the antigen binding domain comprises SEQ ID NO:8 and SEQ ID NO:9, or sequences with at least 95% identity to SEQ ID NOs: 8 and 9.

11. The receptor of claim 7, wherein the antigen binding domain comprises SEQ ID NO:8 and SEQ ID NO:9 or 17, or sequences with at least 95% identity to SEQ ID NOs: 8 and 9, provided SEQ ID NO:8 comprises the three CDRs DRGSQS, IYSNGD and AVNFGGGKLIF and provided SEQ ID NO:9 comprises the three CDRs MRHNA, SNTAGT and ASSLSFGTEAFF.

12. The receptor of claim 1, wherein the antigen binding domain comprises one constant domain represented by SEQ ID NO:3 or sequences with at least 98% identity to SEQ ID NO:3 provided the amino acid residues in position 48 and 91 are both cysteine residues; and one constant domain represented by SEQ ID NO:4 or sequences with more than 98% identity to SEQ ID NO:4 provided the amino acid residues in position 57 and 131 are both cysteine residues.

13. The receptor of claim 2, comprising a constant domain from an alpha chain and a constant domain from a beta chain; wherein the threonine residue in position 48 in the constant domain from the alpha chain is substituted with a cysteine residue and wherein the serine residue in position 57 in the constant domain from the beta chain is substituted with a cysteine residue.

14. A chimeric antigen receptor (CAR), comprising:
a) an extracellular antigen binding domain that specifically binds to HLA complexes presenting peptides, wherein said extracellular antigen binding domain comprises two polypeptides, wherein each of said polypeptides comprises a variable domain and a constant domain, wherein said two polypeptides are connected by at least two disulfide bridges, each bridge formed by two cysteine residues in the constant domains of said polypeptides;
b) a single transmembrane domain operably linked to said extracellular antigen binding domain; and
c) an intracellular signaling domain operably linked to said transmembrane domain.

15-26. (canceled)

27. A nucleic acid encoding the receptor of claim 1.

28-29. (canceled)

30. A cell expressing the receptor of claim 1 in its cell membrane.

31. The cell of claim 30, wherein said cell is selected from the group consisting of T cell, a natural killer cell, and a NK-92 cell.

32. The cell of claim 30, further expressing in its cell membrane a conventional CAR targeting surface epitopes via antigen binding domains from antibodies.

33. (canceled)

34. A method for stimulating a lymphocyte-mediated immune response to a target cell population or tissue in a subject, the method comprising administering to a subject an effective amount of a cell of claim 30.

35. The method of claim 34, wherein said target cell population or tissue is a cancer cell or tumor.

36-39. (canceled)


SEQUENCE LISTING (31 sequences)

SEQ ID NO: 1 - protein, 111 aa, Artificial Sequence
  Synthetic
GIQVEQSPPDLILQEGANSTLRCNFSDSVNNLQWFHQNPWGQLINLFYIPSGTKQNGRLSATTVATERYSLLYISSSQTTDSGVYFCAVNAGNMLTFGGGTRLMVKPHIQN

SEQ ID NO: 2 - protein, 131 aa, Artificial Sequence
  Synthetic
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSSGVTGELFFGEGSRLTVL

SEQ ID NO: 3 - protein, 91 aa, Artificial Sequence
  Synthetic
PDPAVYQLRDSKSSDKSVCLFTDFDSQTNVSQSKDSDVYITDKCVLDMRSMDFKSNSAVAWSNKSDFACANAFNNSIIPEDTFFPSPESSC

SEQ ID NO: 4 - protein, 131 aa, Artificial Sequence
  Synthetic
EDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADC

SEQ ID NO: 5 - protein, 27 aa, Artificial Sequence
  Synthetic
FWVLVVVGGVLACYSLLVTVAFIIFWV

SEQ ID NO: 6 - protein, 41 aa, Artificial Sequence
  Synthetic
RSKRSRLLHSDYMNMTPRRPGPTRKHYQPYAPPRDFAAYRS

SEQ ID NO: 7 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 114 aa, Artificial Sequence
  Synthetic
QKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQN

SEQ ID NO: 9 - protein, 131 aa, Artificial Sequence
  Synthetic
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVV

SEQ ID NO: 10 - protein, 91 aa, Artificial Sequence; identical to SEQ ID NO: 3
  Synthetic

SEQ ID NO: 11 - protein, 131 aa, Artificial Sequence
  Synthetic
EDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADC

SEQ ID NO: 12 - protein, 21 aa, Artificial Sequence
  Synthetic
LLAGLVAADAVASLLIVGAVF

SEQ ID NO: 13 - protein, 24 aa, Artificial Sequence
  Synthetic
LCARPRRSPAQEDGKVYINMPGRG

SEQ ID NO: 14 - protein, 21 aa, Artificial Sequence
  Synthetic
GVLAGIVMGDLVLTVLIALAV

SEQ ID NO: 15 - protein, 39 aa, Artificial Sequence
  Synthetic
YFLGRLVPRGRGAAEAATRKQRITETESPYQELQGQRSD

SEQ ID NO: 16 - protein, 112 aa, Artificial Sequence
  Synthetic
DVKVTQSSRYLVKRTGEKVFLECVQDMDHENMFWYRQDPGLGLRLIYFSYDVKMKEKGDIPEGYSVSREKKERFSLILESASTNQTSMYLCASSSGVTGELFFGEGSRLTVL

SEQ ID NO: 17 - protein, 112 aa, Artificial Sequence
  Synthetic
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVV

SEQ ID NO: 18 - protein, 108 aa, Artificial Sequence
  Synthetic
QSVAQPEDQVNVAEGPLTVKCTYSVSGPYLFWYVQYPRGLQFLLKYITGDLVKGSYGFEAEFKSQTSFHLKKPSALVSDSALYFCAVEDARLMFGDGTQLVVKPNIQN

SEQ ID NO: 19 - protein, 110 aa, Artificial Sequence
  Synthetic
DTAVSQTPKYLVTQMGDKSIKCEQNLGHDTMYWYKQDSKKFLKTMFSYNNKELIINETVPRFSPKSPDKAHLLHINSLELGDSAVYFCASSQLADYNEQFFGPGTRLTVL

SEQ ID NO: 20 - protein, 90 aa, Artificial Sequence
  Synthetic
PDPAVYQLRDSKSSDKSVCLFTDFDSQTNVSQSKDSDVYITDKCVLDMRSMDFKSNSAVAWSKSDFACANAFNNSIIPEDTFFPSPESSC

SEQ ID NO: 21 - protein, 127 aa, Artificial Sequence
  Synthetic
EDLKNVFPPEVAVFEPSEAEISHTQKATLVCLATGFYPDHVELSWWVNGKEVHSGVCTDPQPLKEQPALDSRYCLSSRLRVSATFWQPRHFRCQVQFYGLSEDEWTQDRAKPVTQIVSAEAWGRADC

SEQ ID NO: 22 - protein, 690 aa, Artificial Sequence
  Synthetic
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVNAGNMLTFGGGTRLMVKPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCRAKRGSGATNFSLLKQAGDVEENPGPMGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSSGVTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 691 aa, Artificial Sequence
  Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCRAKREGRGSLLTCGDVEENPGPMRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 695 aa, Artificial Sequence
  Synthetic
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVEDARLMFGDGDGTQLVVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCRAKRGSGATNFSLLKQAGDVEENPGPMGFRLLCCGAFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQLADYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 556 aa, Artificial Sequence
  Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCRAKREGRGSLLTCGDVEENPGPMRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCLLAGLVAADAVASLLIVGAVFLCARPRRSPAQEDGKVYINMPGRG

SEQ ID NO: 26 - protein, 571 aa, Artificial Sequence
  Synthetic
MMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCRAKREGRGSLLTCGDVEENPGPMRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGVLAGIVMGDLVLTVLIALAVYFLGRLVPRGRGAAEAATRKQRITETESPYQELQGQRSD

SEQ ID NO: 27 - DNA, 40 bp, Artificial Sequence
  Synthetic
ggtagagcagactgtggtaaattttgggtgctggtggtgg

SEQ ID NO: 28 - DNA, 36 bp, Artificial Sequence
  Synthetic
ctcgagttagcgaggaggcagggcctgcatgtgaag

SEQ ID NO: 29 - DNA, 17 bp, Artificial Sequence
  Synthetic
caccatgaagaggatat

SEQ ID NO: 30 - DNA, 17 bp, Artificial Sequence
  Synthetic
caccatgatgaaatcct

SEQ ID NO: 31 - DNA, 41 bp, Artificial Sequence
  Synthetic
ccaccaccagcacccaaaatttaccacagtctgctctaccc